(19) United States
(12) Patent Application Publication
(10) Pub. No.: US 2023/0307886 A1
(43) Pub. Date: Sep. 28, 2023

(54) WAVELENGTH STABILIZATION OF PULSED LASERS

(71) Applicant: Open Water Internet Inc., San Francisco, CA (US)

(72) Inventors: Soren Konecky, Alameda, CA (US); Hosain Haghany, San Francisco, CA (US); Albert P. Heberle, Santa Clara, CA (US)

(21) Appl. No.: 18/126,855

(22) Filed: Mar. 27, 2023

Related U.S. Application Data
(60) Provisional application No. 63/324,279, filed on Mar. 28, 2022.

(51) Int. Cl. H01S 5/068; H01S 5/06; G02B 27/48; A61B 5/026

(52) U.S. Cl. CPC H01S 5/06821; H01S 5/0612; G02B 27/48; A61B 5/0261; G01N 2021/479

(57) ABSTRACT

To achieve wavelength stabilization in pulsed lasers, a laser oscillator and a laser amplifier are driven with currents in a pre-lasing stage and a lasing stage. The laser oscillator is co-packaged with the laser amplifier.

### CROSS-REFERENCE TO RELATED APPLICATIONS

This application claims priority to U.S. provisional Application No. 63/324,279 filed Mar. 28, 2022, which is hereby incorporated by reference.

### TECHNICAL FIELD

This disclosure relates generally to optics, and in particular to lasers.

### BACKGROUND INFORMATION

Continuous Wave (CW) lasers and pulsed lasers are operated in different modes. CW lasers emit a continuous light beam that is usually at the same intensity while the light beam is being emitted. Pulsed lasers emit bursts of light (pulses) for a very short duration (e.g. picoseconds to microseconds in some contexts) followed by an off-time where no pulse is emitted. The off-time may be microseconds or even seconds long. CW lasers tend to have a more stable light beam output than pulsed lasers in terms of intensity and wavelength. However, the power requirements to operate the CW lasers continuously may be prohibitive, in some contexts.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified.

FIG. 1 illustrates a conventional laser system including a laser oscillator and a laser amplifier.

FIG. 2 illustrates a laser device including a 2-section Distributed Bragg Reflector (DBR) laser having a ridge section and a taper section, in accordance with aspects of the disclosure.

FIG. 3 illustrates a monolithic laser device including a laser oscillator and a laser amplifier, in accordance with aspects of the disclosure.

FIG. 4 illustrates interleaving laser oscillator pre-pulses and laser amplifier pre-pulses during a pre-lasing stage before emitting a laser pulse in a lasing stage of operating a laser device, in accordance with aspects of the disclosure.

FIG. 5 shows a laser light output during a lasing stage, in accordance with aspects of the disclosure.

FIG. 6 illustrates an example flow chart for a process for wavelength stabilization in pulsed lasers, in accordance with aspects of the disclosure.

FIG. 7 illustrates an oscillator current driven onto a laser oscillator during a non-pulsing stage and the pulsing stage, in accordance with aspects of the disclosure.

FIG. 8 illustrates reducing the magnitude of a laser oscillator current driven onto the laser oscillator during a pulsing stage, in accordance with aspects of the disclosure.

FIG. 9 illustrates further oscillation frequency reduction of an interferometer measurement in a pulsing stage when the initial oscillator current driven onto the laser oscillator is reduced during the pulsing stage, in accordance with aspects of the disclosure.

FIG. 10 illustrates an optional delaying of the reduction of the laser oscillator current in the pulsing stage, in accordance with aspects of the disclosure.

FIG. 11 illustrates an example flow chart for a process for wavelength stabilization in pulsed lasers, in accordance with aspects of the disclosure.

### DETAILED DESCRIPTION

Embodiments of wavelength stabilization of pulsed lasers are described herein. In the following description, numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In aspects of this disclosure, visible light may be defined as having a wavelength range of approximately 380 nm-700 nm. Non-visible light may be defined as light having wavelengths that are outside the visible light range, such as ultraviolet light and infrared light. Infrared light having a wavelength range of approximately 700 nm-1 mm includes near-infrared light. In aspects of this disclosure, near-infrared light may be defined as having a wavelength range of approximately 700 nm-1.5 μm.

In aspects of this disclosure, the term “transparent” may be defined as having greater than 90% transmission of light. In some aspects, the term “transparent” may be defined as a material having greater than 90% transmission of visible light.

Throughout this specification, several terms of art are used. These terms are to take on their ordinary meaning in the art from which they come, unless specifically defined herein or the context of their use would clearly suggest otherwise.

Due to relatively short pulse durations in pulses generated by pulsed lasers, the wavelength of the pulses vary compared to the more steady wavelength of continuous wave (CW) lasers. The wavelength variations may be at least partially attributable to the thermal ramping from when the pulsed laser is off and when the pulsed laser emits the pulse. In contexts such as signal measurement, the wavelength instability increases the linewidth of the laser pulse to an unacceptable level for some measurements.

In the particular example of speckle contrast measurements, wavelength stability may be particularly important. One practical use-case of speckle contrast measurements is detecting blood flow from changes in speckle contrast. In implementations of the disclosure, a laser pulse may be directed into tissue. An exit signal of the laser pulse exiting the tissue is then measured by a photodiode or an image sensor. A change in the speckle contrast of the exit signal is then used to determine the blood flow in the tissue. The laser pulses for this measurement may be 100 microseconds or less. In some implementations, the laser pulses are less than 150 microseconds. In some implementations, the laser pulses are less than 500 microseconds. Since the speckle contrast decreases for longer laser pulses when the light is scattered multiple times by tissue, shorter laser pulses may be advantageous for speckle contrast measurements. Furthermore, the shorter pulses may also limit the light energy of the pulses directed into the tissue. Yet another potential advantage of the short pulses (having wavelength stability) is they expand the design freedom for devices that may perform blood flow measurements, since the shorter pulses translate into less overall power and therefore the potential ability to integrate the pulsed laser and speckle contrast measurement into a wearable device.

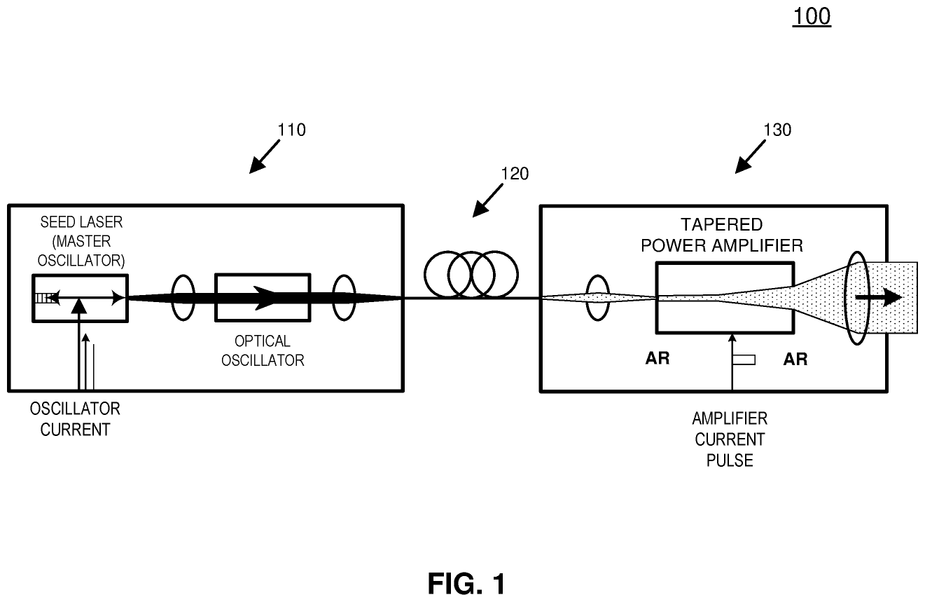

FIG. 1 illustrates a conventional laser system 100 including a laser oscillator (seed laser) 110 and a laser amplifier 130. The laser oscillator 110 and the laser amplifier 130 are optically coupled via a fiber 120. The size of the conventional laser system 100 is not suitable for some contexts. For example, conventional laser system 100 would likely be too large to implement in a wearable device. Additionally, the laser system 100 is costly to manufacture at scale.

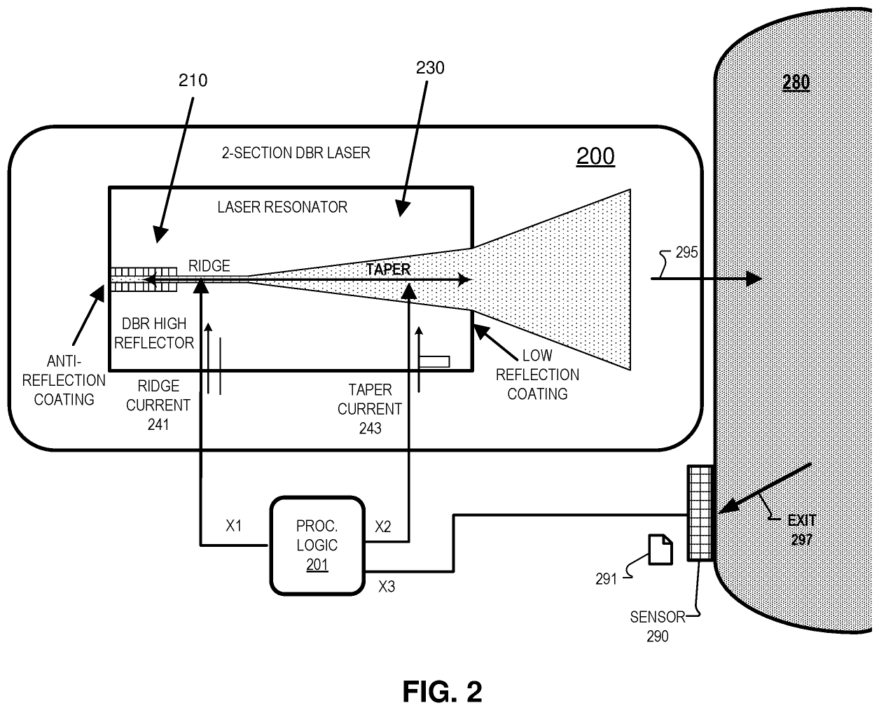

FIG. 2 illustrates laser device 200 including a 2-section Distributed Bragg Reflector (DBR) laser having a ridge section 210 and a taper section 230, in accordance with aspects of the disclosure. Ridge section 210 may also be referred to as a laser oscillator. Processing logic 201 is configured to drive ridge section 210 with output X1 and configured to drive taper section 230 with output X2. Processing logic 201 may drive a first electrical current (ridge current 241) through ridge section 210 and drive a second electrical current (taper current 243) through taper section 230 to emit laser light 295 from laser device 200. Since the laser resonator includes the taper, taper pulsing (heating) modulates the laser resonator (ridge section 210).

In FIG. 2, the laser light 295 is directed into tissue 280. The laser light 295 may be a laser pulse, in accordance with implementations of the disclosure. Laser light 295 may be near-infrared light. Laser light 295 scatters in tissue 280 and a portion of laser light 295 is received by sensor 290 as exit signal 297. Processing logic 201 may receive sensor data 291 by way of input/output X3. Sensor 290 may include an image sensor or one or more photodiodes. Sensor data 291 may include an image captured by an image sensor or analog or digital data. Processing logic 201 may execute speckle contrast detection techniques on sensor data 291 in order to determine a laser speckle value of exit signal 297. The laser speckle value from tissue 280 may be indicative of blood flow, and the laser intensity value from tissue 280 may be indicative of blood volume, and/or blood oxygenation data, for example.

In implementations where laser speckle is analyzed by processing logic 201, coherent light interference in an image (e.g. an image included in sensor data 291) may be manifest or captured as speckles, which include bright and dark spots of one or more pixels in an image. Dark pixels are pixels that have a lower pixel value than surrounding pixels and/or than the average pixel value of an image. Bright pixels are pixels that have a higher pixel value than surrounding pixels and/or than the average pixel value of an image. Quantities of speckles, and therefore coherent light interference, in an image may be detected using the standard deviation of all of the pixels of an image. More specifically, speckle contrast may be determined by dividing the standard deviation of the pixel values of an image by the mean of the pixel values of an image (i.e., standard deviation/mean). The speckle contrast of an image is compared to one or more data models that map the speckle contrast to quantities of blood flowing through a tissue sample, in an embodiment. Blood characteristics may include the quantity of blood flowing through an area, the velocity of the blood, and may also include the concentration and oxygenation levels of hemoglobin. Some blood characteristics are blood flow characteristics and blood flow characteristics may include the quantity of blood flowing through a region of tissue and the velocity of blood flowing through a region of tissue. Some blood characteristics may be independent or less dependent on blood flow, and these blood characteristics may include the concentration and oxygenation levels of hemoglobin.

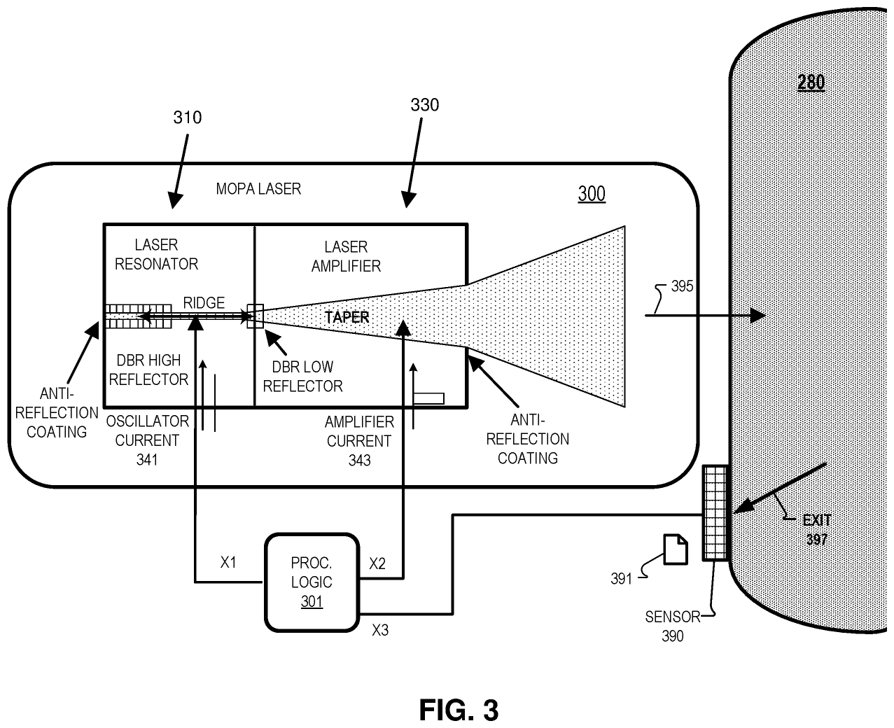

FIG. 3 illustrates laser device 300 including a laser oscillator 310 and a laser amplifier 330, in accordance with aspects of the disclosure. Laser oscillator 310 may also be referred to as a laser resonator or master oscillator and the laser amplifier 330 may be referred to as power amplifier. Taken together, laser oscillator 310 and laser amplifier 330 may be referred to as a Master Oscillator Power Amplifier (MOPA) laser 300. Laser oscillator 310 is co-packaged with power amplifier 330. In some implementations, “co-packaged” means that laser oscillator 310 and power amplifier 330 share a same semiconductor die. In some implementations, “co-packaged” means that laser oscillator 310 and power amplifier 330 are made of separate dies and packaged in a same chip package. Laser oscillator 310 and power amplifier 330 may be thermally isolated from each other even when they are fabricated on a single chip. In laser device 300, the laser oscillator 310 excludes the taper portion and therefore taper pulsing (heating) does not modulate the resonator portion of laser oscillator 310 directly, although some temperature change may be received by laser oscillator 310 from pulsing of laser amplifier 330.

Processing logic 301 is configured to drive laser oscillator 310 with output X1 and configured to drive laser amplifier 330 with output X2. Processing logic 301 may drive a first electrical current (oscillator current 341) through laser oscillator 310 and drive a second electrical current (amplifier current 343) through laser amplifier 330 to emit laser light 395 from laser device 300. The laser light 395 may be near-infrared light. The laser light 395 may be 1067 nm. The laser light 395 may be 785 nm.

In FIG. 3, the laser light 395 is directed into tissue 280. The laser light 395 may be a laser pulse, in accordance with implementations of the disclosure. Laser light 395 scatters in tissue 280 and a portion of laser light 395 is received by sensor 390 as exit signal 397. Processing logic 301 may receive sensor data 391 by way of input/output X3. Sensor 390 may include an image sensor or one or more photodiodes. Sensor data 391 may include an image captured by an image sensor or analog or digital data. Processing logic 301 may execute speckle contrast detection techniques on sensor data 391 in order to determine a laser speckle value of exit signal 397. The laser speckle value from tissue 280 may be indicative of blood flow, and the laser intensity value from tissue 280 may be indicative of blood volume, and/or blood oxygenation data, for example.

In implementations where laser speckle is analyzed by processing logic 301, coherent light interference in an image (e.g. an image included in sensor data 391) may be manifest or captured as speckles, which include bright and dark spots of one or more pixels in an image. Dark pixels are pixels that have a lower pixel value than surrounding pixels and/or than the average pixel value of an image. Bright pixels are pixels that have a higher pixel value than surrounding pixels and/or than the average pixel value of an image. Quantities of speckles, and therefore coherent light interference, in an image may be detected using the standard deviation of all of the pixels of an image. More specifically, speckle contrast may be determined by dividing the standard deviation of the pixel values of an image by the mean of the pixel values of an image (i.e., std/mean). The speckle contrast of an image is compared to one or more data models that map the speckle contrast to quantities of blood flowing through a tissue sample, in an embodiment.

The laser devices 200 and 300 of FIG. 2 and FIG. 3 may be used for continuous wave (CW) laser operation so that the wavelength of the laser light is stable. If laser devices 200 and 300 are used as pulsed-lasers to generate laser pulses in the conventional manner, the wavelength has instability at least in part due to the rise of the temperature of the laser oscillator during the pulsing time period. Laser devices 200 and 300 may be driven in accordance with implementations of the disclosure and included into a wearable device.

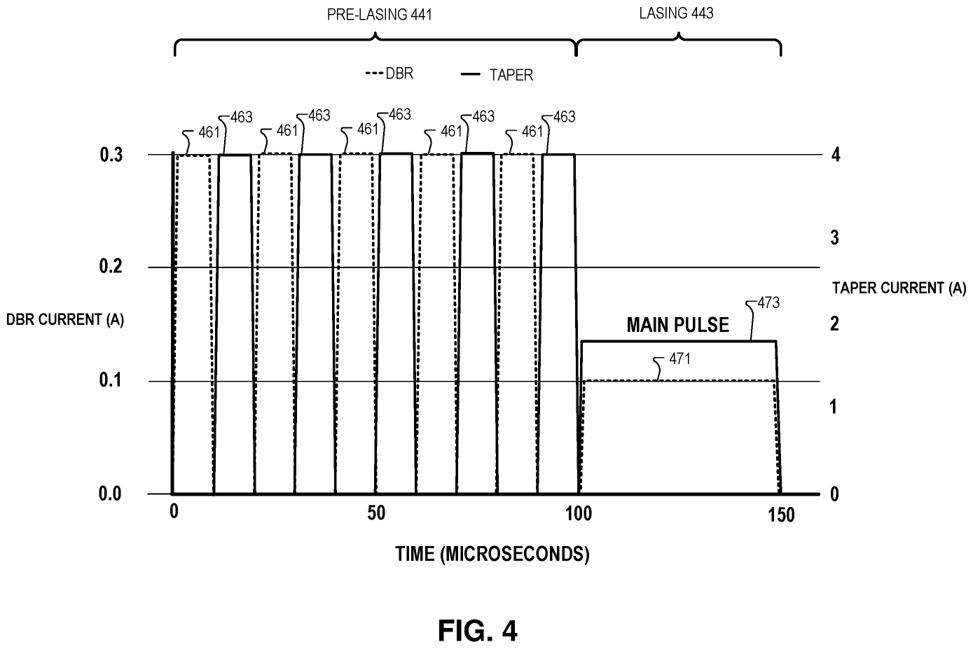

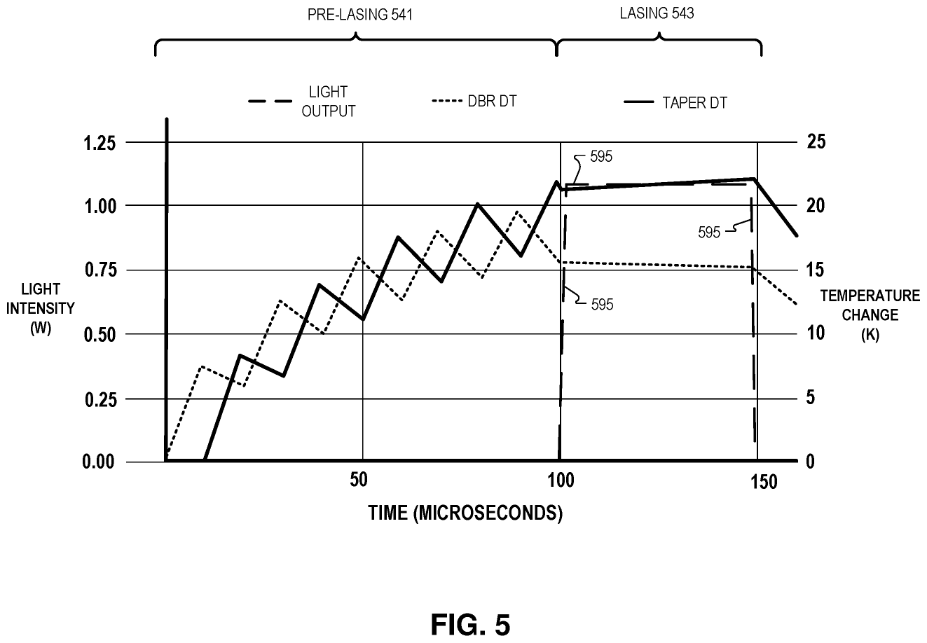

FIGS. 4 and 5 illustrate an example method discovered by Applicant of generating laser pulses with more stabilized wavelengths so that the linewidth of the laser pulses remains narrow. A narrow linewidth for laser pulses improves laser speckle measurements in some contexts. In the context of imaging deep (e.g. more than 1 cm) in tissue, shorter laser pulses (e.g. between 5 μs and 500 μs pulses) yield a larger change in speckle contrast measurements. Hence, the imaging signal is enhanced when the laser pulses are short and have narrow linewidth.

FIG. 4 illustrates interleaving laser oscillator pre-pulses and laser amplifier pre-pulses during a pre-lasing stage 441 before emitting a laser pulse in a lasing stage 443 of operating laser device 300, in accordance with implementations of the disclosure. The dashed-line pulses 461 (“DBR” in the legend) are the laser oscillator pre-pulses in the pre-lasing stage 441 and the solid-line pulses 463 (“TAPER” in the legend) are the laser amplifier pre-pulses in the pre-lasing stage 441. In the pre-lasing stage 441, the laser oscillator pre-pulses 461 and the laser amplifier pre-pulses 463 are not driven high at the same time and thus laser pulses are not emitted during pre-lasing stage 441. In lasing stage 443, laser oscillator pulse 471 is driven simultaneously with laser amplifier pulse 473 to cause a laser pulse to be emitted during the lasing stage 443 that follows the pre-lasing stage 441. In an implementation, processing logic 301 drives laser oscillator pulse 471 onto laser resonator 310 and drives laser amplifier pulse 473 onto laser amplifier 330.

FIG. 5 shows that there is only laser light output (long-dash line) during the lasing stage 543 where a laser pulse 595 is emitted for 50 μs. FIG. 5 shows that when the laser oscillator is pulsed (pre-pulses 461 in FIG. 4) separately from the laser amplifier (pre-pulses 463 in FIG. 4), there is no laser emission but the temperature of the laser oscillator (represented by short-dash line) and the temperature of the laser amplifier (represented by solid line) are increasing during the pre-lasing stage 541. Notably, the temperatures of the laser amplifier and the laser oscillator rise in the pre-lasing stage 541 to approximately the temperature that they will have in the lasing stage 543. Therefore, interleaving the pre-pulses in the pre-lasing stage allows the temperature to rise to what it will be in the lasing stage without actually emitting a laser pulse (only dark pre-pulses) since the laser oscillator and the laser amplifier are not driven at the same time in the pre-lasing stage 541. Stabilizing the temperature at or near the temperature of the lasing stage 443/543 prior to emitting the laser pulse 595 narrows the linewidth of the laser pulse 595 because the laser device (e.g. device 300) is already up to pulsing temperature at the beginning of the lasing stage.

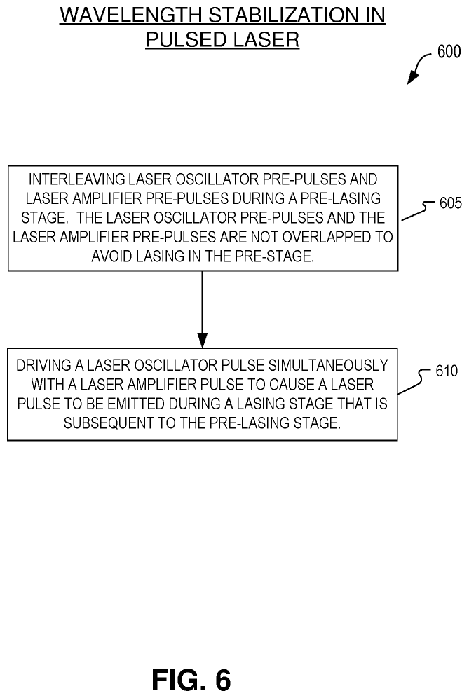

FIG. 6 illustrates an example flow chart for a process 600 for wavelength stabilization in pulsed lasers, in accordance with aspects of the disclosure. The order in which some or all of the process blocks appear in process 600 should not be deemed limiting. Rather, one of ordinary skill in the art having the benefit of the present disclosure will understand that some of the process blocks may be executed in a variety of orders not illustrated, or even in parallel.

In process block 605, laser oscillator pre-pulses (e.g. pre-pulses 461) are interleaved with laser amplifier pre-pulses (e.g. pre-pulses 463) during a pre-lasing stage. The laser oscillator pre-pulses are driven onto a laser oscillator co-packaged with a laser amplifier that receives the laser amplifier pre-pulses. The laser oscillator pre-pulses and the laser amplifier pre-pulses are not overlapped to avoid lasing in the pre-lasing stage.

In process block 610, a laser oscillator pulse (e.g. pulse 471) is driven simultaneously with a laser amplifier pulse (e.g. pulse 473) to cause a laser pulse (e.g. pulse light output 595) to be emitted during a lasing stage that is subsequent to the pre-lasing stage. The laser oscillator pulse is driven onto the laser oscillator and the laser amplifier pulse is driven onto the laser amplifier.

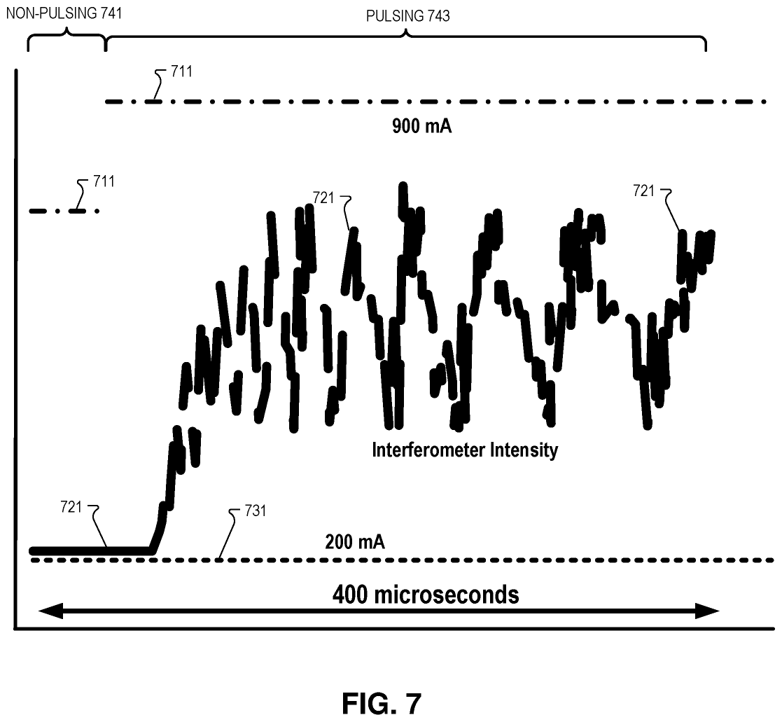

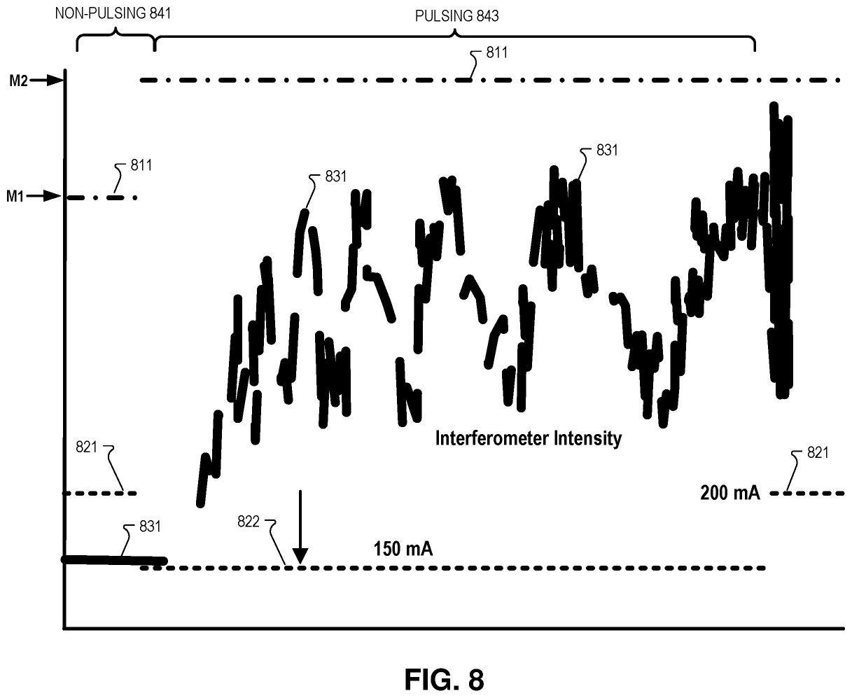

FIG. 7 illustrates a conventional method of operating a laser and FIGS. 8-10 illustrate an example second method discovered by Applicant of generating laser pulses with more stabilized wavelengths. FIG. 7 illustrates an oscillator current 731 of 200 mA driven onto a laser oscillator (e.g. laser oscillator 310) during the non-pulsing stage 741 and the pulsing stage 743. For pulsing stage 743, the laser amplifier current 711 is driven higher (to 900 mA) compared to the non-pulsing stage 741. The oscillation of the interferometer measurement 721 indicates that the wavelength of the output laser light is chirped (wavelength moving up and/or down) during the pulsing stage 743. The wavelength is not desirably stabilized for speckle contrast imaging in FIG. 7.

FIG. 8 illustrates reducing the magnitude of a laser oscillator current (represented by dashed line) driven onto the laser oscillator during a pulsing stage 843. In the illustrated example, an initial oscillator current 821 is 200 mA for the non-pulsing stage 841 and that current magnitude is reduced to a reduced oscillator current 822 of 150 mA for the pulsing stage 843. The laser amplifier current 811 (represented by dashed-dot line) driven onto the laser amplifier is increased from the non-pulsing stage 841 to pulsing stage 843. In FIG. 8, the laser amplifier current 811 driven onto the laser amplifier is increased from magnitude M1 in the non-pulsing stage 841 to magnitude M2 in pulsing stage 843. The oscillation frequency of the interferometer measurement 831 of the laser output is reduced in pulsing stage 843 compared with FIG. 7 indicating that the wavelength change during the measurement shown in FIG. 8 is less than the wavelength change during the measurement shown in FIG. 7.

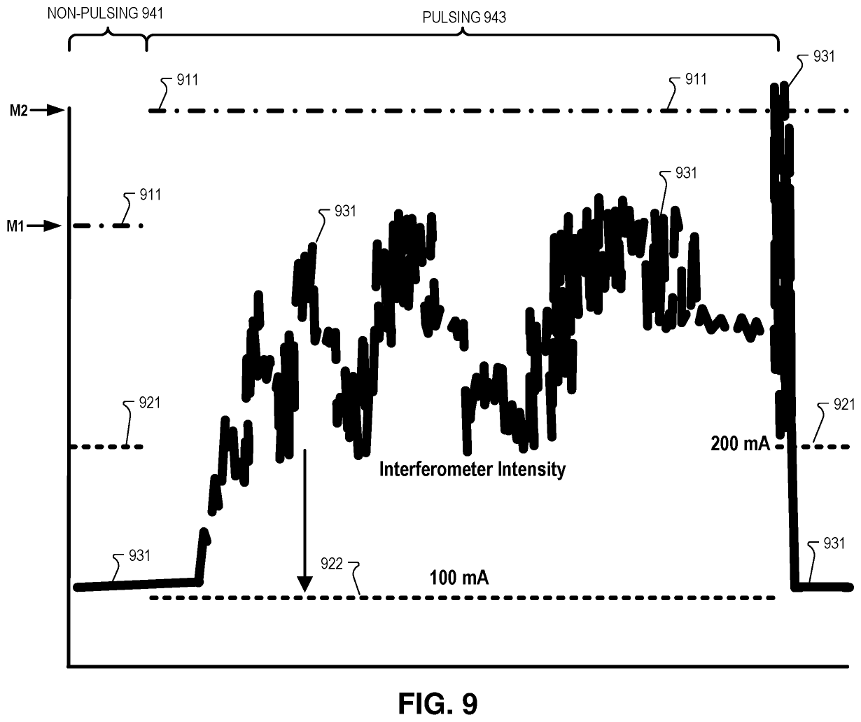

FIG. 9 illustrates further oscillation frequency reduction of the interferometer measurement 931 in a pulsing stage 943 when the initial oscillator current 921 driven onto the laser oscillator is reduced from 200 mA to 100 mA during the pulsing stage 943. In this implementation, the initial oscillator current 921 is reduced 50% to the pulsing oscillator current 922 during pulsing stage 943. In other implementations, the reduced current level driven onto the laser oscillator during pulsing stage 943 is less than 60 percent of the electrical current driven onto the laser oscillator during the non-pulsing stage 941. In other implementations, the reduced current level driven onto the laser oscillator during pulsing stage 943 is less than 50 percent of the electrical current driven onto the laser oscillator during the non-pulsing stage 941. In FIG. 9, the laser amplifier current driven onto the laser amplifier is increased from a non-pulsing current magnitude (M1) in the non-pulsing stage 941 to a pulsed current magnitude (M2) in the pulsing stage 943.

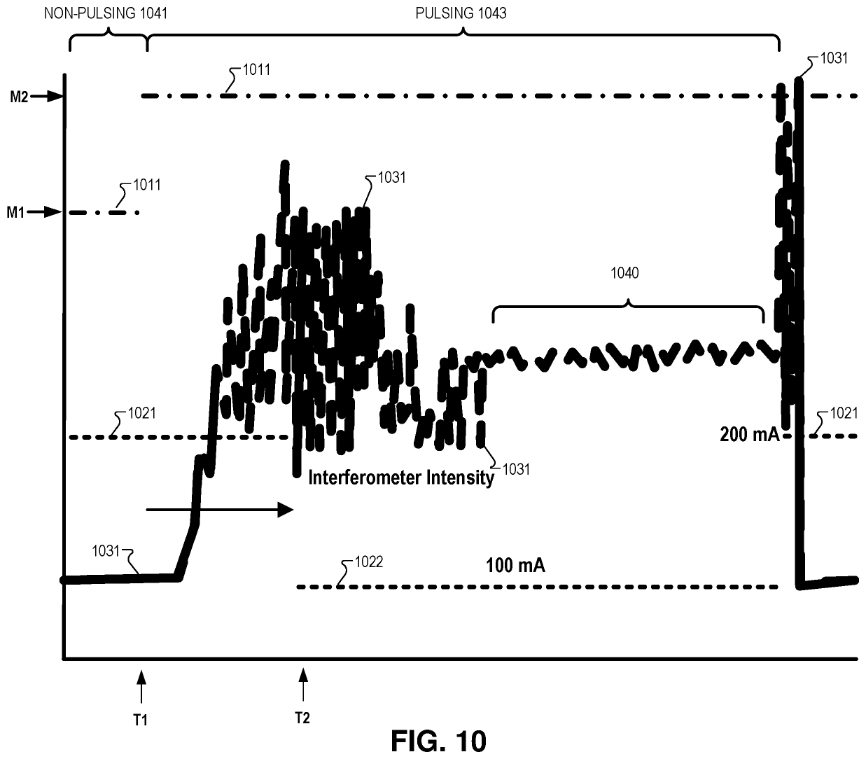

FIG. 10 illustrates an optional delaying of the reduction of the laser oscillator current in the pulsing stage, in accordance with aspects of the disclosure. In FIG. 8 and FIG. 9, the laser oscillator current was reduced at the same time the laser amplifier current was increased. In FIG. 10, the laser amplifier current 1011 is increased from non-pulsing current magnitude (M1) to pulsing current magnitude M2 at starting period (T1) of pulsing stage 1043. However, the reduction of the initial oscillator current 1021 is delayed until a second time period (T2) of the pulsing stage 1043. In the illustrated example, the second time period T2 may be delayed from the starting period T1 by approximately 25% of the time of pulsing stage 1043. The second time period T2 may be delayed from the starting period T1 by 20% or more of the pulsing stage in other implementations. Notably, a stabilized portion 1040 of the interferometer measurement 1031 in pulsing stage 1043 has a much improved wavelength stabilization signal having very low chirp that can yield large improvements in speckle contrast imaging.

Similar to FIG. 9, FIG. 10 shows the initial oscillator current 1021 driven onto the laser oscillator is reduced from 200 mA to 100 mA during the pulsing stage 1043. In this implementation, the initial oscillator current 1021 is reduced 50% to the pulsing oscillator current 1022 during pulsing stage 1043. In other implementations, the reduced current level driven onto the laser oscillator during pulsing stage 1043 is less than 60 percent of the electrical current driven onto the laser oscillator during the non-pulsing stage 1041. In other implementations, the reduced current level driven onto the laser oscillator during pulsing stage 1043 is less than 50 percent of the electrical current driven onto the laser oscillator during the non-pulsing stage 1041. In FIG. 10, the laser amplifier current driven onto the laser amplifier is increased from a non-pulsing current magnitude (M1) in the non-pulsing stage 1041 to a pulsed current magnitude (M2) in the pulsing stage 1043.

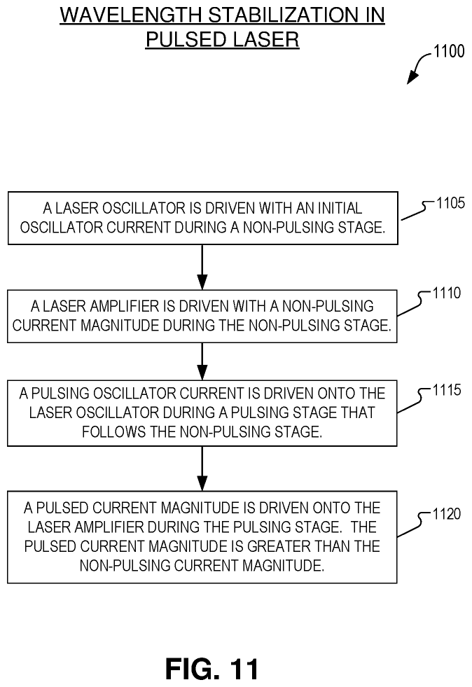

FIG. 11 illustrates an example flow chart for a process 1100 for wavelength stabilization in pulsed lasers, in accordance with aspects of the disclosure. The order in which some or all of the process blocks appear in process 1100 should not be deemed limiting. Rather, one of ordinary skill in the art having the benefit of the present disclosure will understand that some of the process blocks may be executed in a variety of orders not illustrated, or even in parallel.

In process block 1105, a laser oscillator is driven with an initial oscillator current during a non-pulsing stage.

In process block 1110, a laser amplifier is driven with a non-pulsing current magnitude during the non-pulsing stage. The oscillator is co-packaged with the laser amplifier.

In process block 1115, a pulsing oscillator current is driven onto the laser oscillator during a pulsing stage that follows the non-pulsing stage.

In process block 1120, a pulsed current magnitude is driven onto the laser amplifier during the pulsing stage. The pulsed current magnitude is greater than the non-pulsing current magnitude.

In an implementation of process 1100, the non-pulsing current magnitude of the laser amplifier is increased to the pulsed current magnitude at a starting period of the pulsing stage and reducing the initial oscillator current to the pulsing oscillator current is at a second period that is delayed from the starting period.

The term “processing logic” (e.g. 201 or 301) in this disclosure may include one or more processors, microprocessors, multi-core processors, Application-specific integrated circuits (ASIC), and/or Field Programmable Gate Arrays (FPGAs) to execute operations disclosed herein. In some embodiments, memories (not illustrated) are integrated into the processing logic to store instructions to execute operations and/or store data. Processing logic may also include analog or digital circuitry to perform the operations in accordance with embodiments of the disclosure.

A “memory” or “memories” described in this disclosure may include one or more volatile or non-volatile memory architectures. The “memory” or “memories” may be removable and non-removable media implemented in any method or technology for storage of information such as computer-readable instructions, data structures, program modules, or other data. Example memory technologies may include RAM, ROM, EEPROM, flash memory, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device.

Networks may include any network or network system such as, but not limited to, the following: a peer-to-peer network; a Local Area Network (LAN); a Wide Area Network (WAN); a public network, such as the Internet; a private network; a cellular network; a wireless network; a wired network; a wireless and wired combination network; and a satellite network.

Communication channels such as X1 and X2 may include or be routed through one or more wired or wireless communication utilizing IEEE 802.11 protocols, SPI (Serial Peripheral Interface), I2C (Inter-Integrated Circuit), USB (Universal Serial Port), CAN (Controller Area Network), cellular data protocols (e.g. 3G, 4G, LTE, 5G), optical communication networks, Internet Service Providers (ISPs), a peer-to-peer network, a Local Area Network (LAN), a Wide Area Network (WAN), a public network (e.g. “the Internet”), a private network, a satellite network, or otherwise.

A computing device may include a desktop computer, a laptop computer, a tablet, a phablet, a smartphone, a feature phone, a server computer, or otherwise. A server computer may be located remotely in a data center or be stored locally.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible non-transitory machine-readable storage medium includes any mechanism that provides (i.e., stores) information in a form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A device comprising:
a laser comprising:
a laser oscillator configured to emit laser light; and
a laser amplifier co-packaged with the laser oscillator, wherein the laser amplifier is configured to receive the laser light from the laser oscillator; and
driving circuitry configured to:
interleave laser oscillator pre-pulses and laser amplifier pre-pulses during a pre-lasing stage, wherein the laser oscillator pre-pulses are driven onto the laser oscillator and the laser amplifier pre-pulses are driven onto the laser amplifier, and wherein the laser oscillator pre-pulses and the laser amplifier pre-pulses are not overlapped to avoid lasing in the pre-lasing stage; and
driving a laser oscillator pulse simultaneously with a laser amplifier pulse to cause a laser pulse to be emitted during a lasing stage that follows the pre-lasing stage when the laser reaches a wavelength-stabilizing lasing temperature, wherein the laser oscillator pulse is driven onto the laser oscillator and the laser amplifier pulse is driven onto the laser amplifier.

2. The device of claim 1, wherein the laser amplifier pulse is approximately half of a magnitude of the laser amplifier pre-pulses.

3. The device of claim 1, wherein the laser oscillator includes at least one DBR reflector.

4. The device of claim 1, wherein the laser amplifier is a tapered laser amplifier.

5. The device of claim 1, wherein the laser is a near-infrared laser.

6. The device of claim 1, wherein the laser pulse has a time duration between 5 μs and 500 μs.

7. The device of claim 1, wherein the laser oscillator pre-pulses and the laser amplifier pre-pulses are between 1 microsecond and 20 microseconds.

8. The device of claim 1, wherein, at an end of the pre-lasing stage, a laser amplifier temperature and a laser oscillator temperature are approximately the same as a start of the lasing stage.

9. The device of claim 8, wherein approximately the same is within 25%.

10. A method of stabilizing wavelength in a pulsed laser, the method comprising:
interleaving laser oscillator pre-pulses and laser amplifier pre-pulses during a pre-lasing stage, wherein the laser oscillator pre-pulses are driven onto a laser oscillator that is fabricated on a same chip with a laser amplifier that receives the laser amplifier pre-pulses, and wherein the laser oscillator pre-pulses and the laser amplifier pre-pulses are not overlapped to avoid lasing in the pre-lasing stage; and
driving a laser oscillator pulse simultaneously with a laser amplifier pulse to cause a laser pulse to be emitted during a lasing stage that is subsequent to the pre-lasing stage, wherein the laser oscillator pulse is driven onto the laser oscillator and the laser amplifier pulse is driven onto the laser amplifier.

11. The method of claim 10, wherein the laser pulse has a time duration between 5 μs and 100 μs.

12. A device comprising:
a laser comprising:
a laser oscillator configured to emit laser light; and
a laser amplifier co-packaged with the laser oscillator, wherein the laser amplifier is configured to receive the laser light from the laser oscillator; and
driving circuitry configured to:
during a non-pulsing stage, drive the laser oscillator with an initial oscillator current and drive the laser amplifier with a non-pulsing current magnitude; and
during a pulsing stage that follows the non-pulsing stage, reduce the initial oscillator current to a pulsing oscillator current and increase the non-pulsing current magnitude to a pulsed current magnitude.

13. The device of claim 12, wherein the non-pulsing current magnitude of the laser amplifier is increased to the pulsed current magnitude at a starting period of the pulsing stage, and wherein the reducing the initial oscillator current to the pulsing oscillator current is at second period that is delayed from the starting period.

14. The device of claim 13, wherein the second period is delayed from the starting period by more than 20% of the pulsing stage.

15. The device of claim 12, wherein the pulsing oscillator current driven onto the laser oscillator is less than 60 percent of the initial oscillator current.

16. The device of claim 12, wherein the pulsing stage has a time duration between 5 μs and 500 μs.

17. A method of stabilizing wavelength in a pulsed laser, the method comprising:
driving a laser oscillator with an initial oscillator current during a non-pulsing stage;
driving a laser amplifier with a non-pulsing current magnitude during the non-pulsing stage, wherein the laser oscillator is fabricated on a same chip as the laser amplifier;
driving a pulsing oscillator current onto the laser oscillator during a pulsing stage that follows the non-pulsing stage, wherein the pulsing oscillator current is reduced from the initial oscillator current; and
driving a pulsed current magnitude onto the laser amplifier during the pulsing stage, wherein the pulsed current magnitude is greater than the non-pulsing current magnitude.

18. The method of claim 17, wherein the non-pulsing current magnitude of the laser amplifier is increased to the pulsed current magnitude at a starting period of the pulsing stage, and wherein reducing the initial oscillator current to the pulsing oscillator current is at second period that is delayed from the starting period.

19. The method of claim 18, wherein the second period is delayed from the starting period by more than 20% of the pulsing stage.

20. The method of claim 17, wherein the pulsing oscillator current driven onto the laser oscillator is less than 60 percent of the initial oscillator current.
